(19) United States
(12) Patent Application Publication
(10) Pub. No.: US 2021/0010092 A1
(43) Pub. Date: Jan. 14, 2021

(54) GENE RELEVANT TO PAPILLARY THYROID TUMORS

(71) Applicant: BOZHOU XINJIANKANG SCIENCE AND TECHNOLOGY CO., LTD, BOZHOU CITY (CN)

(72) Inventor: LIANGZHONG XIONG, BOZHOU CITY (CN)

(21) Appl. No.: 17/032,414

(22) Filed: Sep. 25, 2020

Related U.S. Application Data
(63) Continuation-in-part of application No. 15/810,383, filed on Nov. 13, 2017.
Application No. 15/810,383 is a continuation of application No. PCT/CN2015/078872, filed on May 13, 2015.

(51) Int. Cl. C12Q 1/6886

(52) U.S. Cl. CPC C12Q 1/6886; C12Q 2600/158

(57) ABSTRACT

The invention relates to a gene relevant to papillary thyroid tumors and an application thereof. According to the base sequence of the gene, real-time and quantitative PCR (Polymerase Chain Reaction) primers are designed and synthesized; the expression level of long-chain non-coding RNA (Ribonucleic Acid) transcribed by the gene is detected in a papillary thyroid carcinoma clinical case specimen; the result shows remarkable reducing of the expression level of the long-chain non-coding RNA in papillary thyroid tumor tissues and the long-chain non-coding RNA of the gene silencing can remarkably promote the growth of thyroid cancer cells. The gene relevant to the papillary thyroid tumors is expected to prepare preparations used in papillary thyroid carcinoma auxiliary diagnosis, gene therapy, curative effect prediction or prognosis.

### CROSS-REFERENCE TO RELATED PATENT APPLICATION

This application is a continuation-in-part of U.S. patent application Ser. No. 15/810,383, filed on Nov. 13, 2017, which itself is a continuation application of International Patent Application No. PCT/CN2015/078872, filed on May 13, 2015. The disclosure of each of the above applications is incorporated herein in its entirety by reference.

Some references, which may include patents, patent applications and various publications, are cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference were individually incorporated by reference.

### FIELD

The present invention relates to the field of molecular biology, and in particular, to a gene relevant to papillary thyroid tumors and a PCR detection method thereof.

### BACKGROUND

Thyroid carcinoma, as the most common endocrine tumor, has become one of the most common malignant tumors since its incidence increases rapidly in recent years, and the incidence of thyroid carcinoma is increasing faster than any other solid tumor. 90% of the thyroid carcinoma is papillary thyroid carcinoma (papillary thyroid cancer, PTC). Compared with other malignant tumors, papillary thyroid carcinoma has a lower mortality but a higher metastasis rate, with a lymph node metastasis rate up to 30-50%. In the event that papillary thyroid carcinoma is spread and recurred but the patient fails to get timely diagnosis and thus loses the optimal operation opportunity, then the mortality is increased significantly and is an important index of poor prognosis.

Epidemiological evidences show that, the female papillary thyroid carcinoma in many places of China has a higher incidence than breast carcinoma and rank the first place. The occurrence of papillary thyroid carcinoma is a multi-gene, multi-step pathological change process, including a series of changes, such as genetics. Etiological study reveals that this malignant tumor contributes most to genetic factors. There is increasing evidences suggesting that oncogene activation and anti-oncogene inactivation due to somatic cell genetic mutations play an essential role in the formation and development of tumors, and therefore, screening on somatic cell mutations of papillary thyroid carcinoma will contribute to finding pathogenic driver genes thereof.

Currently, the diagnosis of thyroid carcinoma mainly depends on thyroid ultrasonography and puncture biopsy histopathology. Puncture biopsy histopathology diagnosis on thyroid carcinoma has been widely used clinically, however, there are still 30% of suspicious or uncertain diagnosis results for thyroid carcinoma, and besides, the puncture is an invasive examination and is painful to the patient. The thyroid carcinoma is generally treated surgically, and the five-year survival rate of a patient at an early stage can reach 95%, the five-year survival rate of a patient at an advanced stage is reduced to 59%, and it is therefore necessary to seek a new diagnosis method which is less invasive and has a higher sensitivity and also a higher specificity, so as to improve the diagnostic accuracy and the early diagnostic rate of thyroid carcinoma.

Due to the complexity of tumor heredity, a conventional somatic cell mutation screening method cannot realize a whole understanding of tumors. However, as a high-efficient and high-sensitivity technology, whole genome exome sequencing can find most of disease-related variations of the exon region, common variants and the low-frequency (<5% frequency) mutations can be detected. Determining gene mutation of the exon region facilitates to determine oncogene and anti-oncogene related to thyroid carcinoma and provide basis for early molecular diagnosis on the one hand, on the other hand, a better qualitative of tumor is facilitated, to reveal sub-clinical classification of similar tissue pathological types and different clinical features, and help to determine the treatment sensitivity and the judgment prognosis of the papillary thyroid carcinomas with different subtypes; but even more critical, exome sequencing technology is helpful in finding the optimal drug target related to the gene mutation of the papillary thyroid carcinoma, so as to change corresponding molecular regulating networks and relevant metabolic pathways, and make individual treatment of the papillary thyroid carcinoma possible.

All or a portion of references, works, patents and patent applications cited in the present invention are explicitly and individually incorporated herein by reference.

### SUMMARY

One object of the present invention is to provide a gene, named GAS8-AS1, having a sequence represented by a sequence listing SEQ ID NO:1.

Another object of the present invention is to provide use of the GAS8-AS1 gene for preparing reagents used in screening, detection or auxiliary diagnosis of papillary thyroid carcinoma.

Another object of the present invention is to provide a nucleic acid molecule hybridizing with the GAS8-AS1 gene under stringent conditions, wherein the nucleic acid molecule is used to prepare reagents for detecting the GAS8-AS1 gene.

In a preferred embodiment of the present invention, the nucleic acid molecule has a sequence represented by SEQ ID NO:2 or SEQ ID NO:3.

Another object of the present invention is to provide a detection kit including the nucleic acid molecule hybridizing with the GAS8-AS1 gene under stringent conditions, wherein the kit is used to detect the GAS8-AS1 gene.

In a preferred embodiment of the present invention, the kit is a real-time and quantitative PCR detection kit.

In a preferred embodiment of the present invention, the nucleic acid molecule included in the kit has a sequence represented by SEQ ID NO:2 or SEQ ID NO:3.

Another object of the present invention is to provide use of the GAS8-AS1 gene as an auxiliary diagnosis marker for papillary thyroid carcinoma.

Another object of the present invention is to provide use of the GAS8-AS1 gene as a therapeutic target in papillary thyroid carcinoma.

### BRIEF DESCRIPTION OF THE DRAWINGS

Further objects, functions and advantages of the present invention will be elucidated with reference to the embodiments described hereinafter and the accompanying drawings in which:

FIG. 1 is a schematic diagram illustrating mutations of lncRNA GAS8-AS1 gene and genes related thereof;

FIG. 2 is an RNA secondary structural diagram of lncRNA GAS8-AS1 predicted by the RNAfold software;

FIG. 3 is a schematic diagram illustrating expression levels of lncRNA GAS8-AS1 in papillary thyroid carcinoma tissues and para-carcinoma normal tissues detected by Zhejiang Queue and Huai'an Queue;

FIG. 4 is a schematic diagram illustrating proliferation of papillary thyroid carcinoma cell lines GLAG66, NPA and TPC-1 after transfecting plasmids carrying lncRNA GAS8-AS1 gene;

FIG. 5 is a schematic diagram illustrating expression levels of lncRNA GAS8-AS1 in papillary thyroid carcinoma cell lines GLAG66, NPA and TPC-1 after transfecting plasmids carrying lncRNA GAS8-AS1 gene by a real-time and quantitative PCR detection;

FIG. 6 is a schematic diagram illustrating proliferation of papillary thyroid carcinoma cell lines GLAG66 and TPC-1 after transfecting siRNAs directed against lncRNA GAS8-AS1; and

FIG. 7 is a schematic diagram illustrating expression levels of lncRNA GAS8-AS1 in papillary thyroid carcinoma cell lines GLAG66 and TPC-1 after transfecting siRNAs directed against lncRNA GAS8-AS1 by a real-time and quantitative PCR detection.

FIG. 8 is a diagram illustrating the inhibition rate of over expression of lncRNA GAS8-AS1 regarding inhibiting the growth of papillary thyroid carcinoma cells collected from papillary thyroid carcinoma patients.

### DETAILED DESCRIPTION

The above contents of the present invention will be explained and described in further details through specific embodiments hereinafter, so that persons skilled in the art will readily understand the present invention, however, the scope of the subject matter described herein should not be constructed as being limited to the following examples, or a limit to any or all claims of the present invention, or to depart from the spirit of the present invention.

The articles “a”, “an”, and “the” as used herein and in the appended claims are used herein to refer to one or to more than one (i.e., to at least one) of the grammatical object of the article unless the context clearly indicates otherwise. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of”.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of and “consisting essentially of shall be closed or semi-closed transitional phrases, respectively, as set forth in the United States Patent Office Manual of Patent Examining Procedures, Section 2111.03.

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a nonlimiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, in certain methods described herein that include more than one step or act, the order of the steps or acts of the method is not necessarily limited to the order in which the steps or acts of the method are recited unless the context indicates otherwise.

The term “patient” or “subject” is used throughout the specification to describe an animal, including human, nonhuman primates (e.g., ape or monkey) or a wild or domesticated animal, to whom treatment, including prophylactic treatment, with the compositions according to the present disclosure is provided. For treatment of those infections, conditions or disease states which are specific for a specific animal such as a human patient, the term patient refers to that specific animal, including a wild or domesticated animal, such as a camelid (e.g. camels, alpacas, or llamas), a dog, a cat, a mouse, a hamster, or a farm animal such as a horse, cow, sheep, donkey, pig, chicken, etc. In general, in the present disclosure, the term patient refers to a human patient unless otherwise stated or implied from the context of the use of the term.

As used herein, the terms “biological sample” or “patient sample” or “test sample” or “sample” as used herein, refer to a sample obtained from an organism or from components (e.g., cells) of a subject or patient for the purpose of diagnosis, prognosis, or evaluation of a subject of interest. The sample can be, for example, exhale condensate, blood, saliva, or lung tissue. In certain embodiments, such a sample may be obtained for assessing the presence of antibodies specific for coronavirus following a suspected infection or following the vaccination using a vaccine construct of the disclosure.

The term “effective” is used to describe an amount of a compound, composition or component which, when used within the context of its intended use, effects an intended result. The term effective subsumes all other effective amount or effective concentration terms, which are otherwise described or used in the present application.

As used herein, the phrases “therapeutically effective amount” and “prophylactically effective amount” refer to an amount that provides a therapeutic benefit in the treatment, or prevention. The specific amount that is therapeutically effective can be readily determined by ordinary medical practitioner, and may vary depending on factors known in the art.

As used herein, the term “isolated” or “purified” polypeptide or protein or virion or biologically-active portion or vaccine construct thereof is substantially free of cellular material or other contaminating proteins from the cell or tissue source from which the polypeptide is obtained.

One object of the present invention is to provide a gene, named GAS8-AS1, having a sequence represented by a sequence listing SEQ ID NO:1.

The GAS8-AS1 gene (NCBI-GeneID: 750), also known as C16orf3 gene (chromosome 16 open reading frame 3), is located in the intron 2 of human chromosome 16 GAS8 gene. The GAS8-AS1 gene doesn't contain any intron sequence that is transcribed in the opposite direction to the GAS8 gene to generate a long non-coding RNA (lncRNA) that cannot be translated into a protein. Currently, biological functions of the gene and its transcription product are largely unclear.

However, the present invention reflects that the GAS8-AS1 gene relates to papillary thyroid carcinoma. In the papillary thyroid carcinoma cells collected from patients and cultivated in vitro, over expression of GAS8-AS1 gene significantly inhibits the growth of papillary thyroid carcinoma cells. In contrast, silencing of GASB-AS1 gene significantly enhances the growth of the papillary thyroid carcinoma cells in vitro.

The sequence of GAS8-AS1 gene (NCBI Reference Sequence: NR_122031.1) is shown as follows:

| (SEQ ID No. 1) | |
|---|---|
| 1 | acctgcagtc ccagctactg ggcagcctga agcagcagga tggtgtgaac ccaggaggtg |
| 61 | gagcttgcag tgagccgagg tcgcgccacc gcactccagc ctgggccaca cagcgagatt |
| 121 | ccgtcagaat cagttacttt tcgggcacag ccccaggcca cttactgtga gcctttttct |
| 181 | ttctcaacac cacattcccc acagggaaaa cacatttctc acctcaaaag aagacaagac |
| 241 | aacgagcaaa caagaaggag cagcaggagg ggttctgagc cgaggatgcc gggcagacat |
| 301 | gagggagaca cgcacccccg aatccaacca gtgcctcggc acaacgacaa atgtcttcac |
| 361 | gtcacagacc tttagaggct cctgggcaga gcctgaacca gggctcctga ctggtctgtt |
| 421 | tggctcacat ggtgttgaga ttttgccatc actcaatatt cagatttctt ataaatatcc |
| 481 | agatttccag cttctcttgg aaaatcagaa aaaaacagca ctgaactcct aggcccacaa |
| 541 | ggcactcccc agtgaacaga tgaaactgtc ctctgctgcg gggcaggagt ctccaggtca |
| 601 | cccccatccc tccccacctg cctggaccct gaagaagcct tctgagtctg tggctcaacg |
| 661 | tgcgatgtgc agtgcaaggg cctgccccgt agcctgcccc gtaggctgcc ccatagcctg |
| 721 | ccccgtaagc tgccccgtag cctgccccgt aggctgcccc gtaggctcca tggccactgc |
| 781 | cccacaaggc ctgtctccac aggaatggga agcggacagg gagacgggca gcagctcaca |
| 841 | tgctgggaca acgcagtgtt caatccattc tccatccagc agctccagac atctttccag |
| 901 | aacacaaacc tgaccccatc acctctctgc ttagccactg gcttaaactg ccaatggttt |
| 961 | gcctgcatgt aaaataaagc cattctttac cattaaaaaa |

Long non-coding RNA (long non-coding RNA, lncRNA) is a kind of non-coding RNA with a transcript of more than 200 nucleotides in length, it is found in recent researches that, lncRNA is a kind of RNA with important biological functions, participating in various important regulating processes, such as genome imprinting, chromosome silencing, chromatin modification, transcriptional activation, transcriptional interference, and intra-nuclear transport, and playing an important role in regulation and control of life activities, such as cell differentiation and development, gene transcription and translation, genetics and epigenetics.

Another object of the present invention is to provide use of the GAS8-AS1 gene for preparing reagents used in screening, detection or auxiliary diagnosis of papillary thyroid carcinoma.

Another object of the present invention is to provide a nucleic acid molecule hybridizing with the GAS8-AS1 gene under stringent conditions, to prepare reagents for detecting the GAS8-AS1 gene. The nucleic acid molecule has a sequence represented by SEQ ID NO:2 or SEQ ID NO:3.

Another object of the present invention is to provide a detection kit including the nucleic acid molecule hybridizing with the GAS8-AS1 gene under stringent conditions, and the kit can be used to detect the GAS8-AS1 gene. Obviously, after learning a gene sequence represented by SEQ ID NO:1 and a primer sequence represented by SEQ ID NO:2 or SEQ ID NO:3 disclosed in the present invention, persons skilled in the art can prepare other primers and kits for detecting the GAS8-AS1 gene without creative efforts, on the basis of the present invention, and the detection method of the kit includes, but is not limited to the real-time and quantitative PCR method. The kit can include the nucleic acid molecule represented by SEQ ID NO:2 or SEQ ID NO:3 or other molecules hybridizing with the GAS8-AS1 gene under stringent conditions. Optionally, the kit also can include auxiliary reagents required for performing conventional gene detection.

Another object of the present invention is to provide use of the GAS8-AS1 gene as an auxiliary diagnosis marker for papillary thyroid carcinoma. As discussed in the present disclosure, the GAS8-AS1 gene in a patient with papillary thyroid carcinoma has a significant level of mutation. Therefore, detecting the level of mutation can be used as an effective method for early screening and auxiliary diagnosis of papillary thyroid carcinoma.

Another object of the present invention is to provide use of the GAS8-AS1 gene as a therapeutic target in papillary thyroid carcinoma. As discussed in the present disclosure, inducing the GAS8-AS1 gene overexpression in a patient with papillary thyroid carcinoma can significantly inhibit the growth of papillary thyroid carcinoma cells. Therefore, GAS8-AS1 can be used as an effective therapeutic target in papillary thyroid carcinoma.

Another object of the present invention is to illustrate the relationship between the GAS8-AS1 gene and papillary thyroid carcinomaý, as well as the application of the GAS8-AS1 gene. Comparing to the known art, the present invention is more advanced because of the application of the GAS8-AS1 gene disclosed therein. In particular, in the papillary thyroid carcinoma cells collected from patients, over expression of GAS8-AS1 gene significantly inhibits the growth of papillary thyroid carcinoma cells in vitro. In contrast, silencing of the GAS8-AS1 gene significantly enhances the growth of the papillary thyroid carcinoma cells in vitro.

As such, the present invention not only shows that the long non-coding RNA GAS8-AS1can be a biomarker for medical diagnosis, but also provides a novel target for therapies treating papillary thyroid carcinoma so as to improves the effectiveness of the therapies treating papillary thyroid carcinoma.

Another object of the present invention is to provide the GAS8-AS1 gene as a novel target for therapies treating papillary thyroid carcinoma. As disclosed below, over expression of the GAS8-AS1 gene in the papillary thyroid carcinoma patients leads to the inhibition of the papillary thyroid carcinoma cells growth. Therefore, GAS8-AS1 can be an effective target for therapy treating the papillary thyroid carcinoma.

### Embodiment 1: Mutation Sequencing Method

1.1 Acquiring a Tumor Tissue Sample of a Subject

1.2 Genomic DNA Extraction

Get ready an autoclaved mortar, pour into liquid nitrogen for pre-cooling after drying; an appropriate amount of tissue is ground with the mortar under addition of liquid nitrogen, grind into powder then thaw; collect the tissue in the mortar with 800 ìl of PBS solution then put the tissue in a centrifuge tube (1.5 ml); centrifuge at 12000 rpm for 1 min, and then remove the supernatant. Then add 200 ìl of buffer GA and shake to thoroughly suspend.

Add 20 ìl of protease K (20 mg/ml), mix thoroughly, incubate at 56° C. for 2 h, and shake once every 20 min till the tissue is dissolved.

Add 200 ìl of GB buffer and mix uniformly upside down and incubate at 70° C. for 10 min until the solution becomes clear.

Add 200 ìl of absolute alcohol, fully oscillate for 15 sec, then flocculent precipitate should appear.

Transfer the above solution and flocculent precipitate to a CB3 adsorption column, then centrifuge at 12,000 rpm for 30 sec and remove the liquid in the collection tube.

Add 500 ìl of GD to the CB3 adsorption column, centrifuge at 12,000 rpm for 30 sec and remove the liquid in the collection tube.

Add 600 ìl of PW (check whether alcohol has been added before use) to the CB3 adsorption column, centrifuge at 12,000 rpm for 30 sec, and remove the liquid in the collection tube. Repeat this step 2 times.

Centrifuge at 12,000 rpm for 2 min, remove waste liquid, and then stand for a few minutes to dry the residual rinse liquid.

Replace the collection tube, add 50 ìl to 200 ìl of TE to the CB3 adsorption column for dissolving DNA, stand for 5 min at room temperature and centrifuge for 2 min at 12,000 rpm, and store the collected DNA at −20° C. for later use.

1.3 Template Preparation

The solid-phase PCR (Illumina's Hiseq) method is used, that is, the amplification process is carried out on glass slides. High-density forward and reverse primers are covalently linked on these glass slides, and the ratio of templates to primers determines the density of the amplified clusters. The solid-phase PCR can produce one to two hundred million spatially isolated template clusters and provide free ends for universal sequencing primers to initiate sequencing reactions.

1.4 Exome Trapping

High-coverage exon region trapping is performed using the Agilent 50 Mb SureSelectXT Human All Exon V5 kit on human exon liquid-phase targeted sequence enrichment system. The All Exon 50 Mb kit is a human all exon trapping kit jointly developed by the Agilent and the Wellcome Trust Sanger Institute and Gencode consortium. Exon trapped can reach up to 50 Mb. Objects to be trapped: 1) exons found in the GENCODE project (about 12M); 2) exons in the NCBI Consensus CDS database (CCDS, March 2009); 3) miRNAs in the Sanger V13 database; 4) over 300 human non-coding RNAs (e.g., snoRNAs and scaRNAs).

1.5 Targeted Sequencing and Bioinformatics Analysis

The basic principle of Illumina's Hiseq 2000 sequencing is sequencing by synthesis, also referred to as cyclic reversible termination. DNA polymerases, linker primers and four dNTPs with base-specific fluorescent labels are added simultaneously to the reaction system. Since the 3′hydroxyl groups of these dNTPs are chemically protected, only one dNTP can be added to each round of synthesis reaction. After dNTP is added to the synthetic chain, all unused free dNTPs and DNA polymerases will be eluted. Add the buffer required to stimulate the fluorescence, excite the fluorescence signal with a laser, record the fluorescence signal with an optical device, and then convert into sequencing results via computer analysis. After the recording of the fluorescent signal is completed, add chemical reagents to quench the fluorescent signal, remove the 3′ hydroxy protecting group of dNTP, restore the 3′ end viscosity, and continue to polymerize the second nucleotide. This continues until all the template sequences are completely polymerized into double-strand. In this way, make statistics to the results of the fluorescence signals collected in each round, to learn the sequence of DNA fragment on each template. One advantage of this sequencing method is to reduce the time for sample separation and preparation, the read length of the paired end can reach up to 2×50 bp, more than 20 GB of high quality filter data can be obtained after each running, and the running cost is relatively low, and therefore, it is a new generation of sequencing technology with a higher cost performance.

Targeted sequencing of papillary thyroid carcinoma is carried out with the method of constructing Gaussian mixture model, to align the obtained short fragment sequence and the reference sequence (mapping), find mutation (variant calling) and filter and screen the mutation.

### Embodiment 2: Taking Genes Such as GAS8-AS1 as a Target for Screening or Detecting Papillary Thyroid Carcinoma

2.1 Experimental Method

According to the present invention, targeted sequencing of 91 pairs of paired tissues (thyroid carcinoma tissues and peripheral blood samples) of papillary thyroid carcinoma patients is carried out with the whole genome exon technique described in Embodiment 1 to obtain gene mutation. Finally, genes, such as GASB-AS1 are determined to be papillary thyroid carcinoma susceptibility genes for the Chinese Han populations. The paired tissues of papillary thyroid carcinoma patients refer to thyroid carcinoma tissues and peripheral blood samples of the patient, and the Cancer Institute and Hospital of the Chinese Academy of Medical Sciences and Zhejiang Cancer Hospital are entrusted to collect the 91 pairs of tissue samples of papillary thyroid carcinoma patients.

2.2 Experimental Result

2.21 Gene Mutation Frequency Statistics

Next generation sequencing is carried out on 91 pairs of paired tissues of papillary thyroid carcinoma, and the high-frequency mutant genes are shown in Table 1.

2.22 Papillary Thyroid Carcinoma Susceptibility Gene

The high-frequency mutant genes are analyzed with MutsigCV software, and GAS8-AS1 is finally determined to be papillary thyroid carcinoma susceptibility gene for the Chinese Han populations. See Table 1.

TABLE 1
Papillary thyroid carcinoma susceptibility
gene for the Chinese Han populations

| | | | Number | | |
|---|---|---|---|---|---|
| | Number | Number | of non- | | |
| | of | of | coding | | False |
| | non-silent | silent | region | | discovery |
| Gene | mutations¹ | mutations² | mutations³ | P value⁴ | rate⁵ |
| BRAF | 53 | 1 | 0 | <1.0E−16 | <1.0E−16 |
| GAS8-AS1 | 15 | 0 | 0 | <1.0E−16 | <1.0E−16 |

Notes:
¹the number of non-silent mutations in the gene;
²the number of silent mutations in the gene;
³the number of non-coding region mutations in the gene;
⁴calculated by MutSigCV software;
⁵multiple testing correction results.
Embodiment 3: Taking GAS8-AS1 gene detection as a target for screening or detecting papillary thyroid carcinoma

The GAS8-AS1 gene (NCBI-GeneID: 750), also known as C16orf3 gene (chromosome 16 open reading frame 3), is located in the intron 2 of human chromosome 16 GAS8 gene. The GAS8-AS1 gene doesn't contain any intron sequence that is transcribed in the opposite direction to the GAS8 gene to generate a long non-coding RNA (lncRNA) that cannot be translated into a protein. Currently, biological functions of the gene and its transcription product are still unclear.

The sequence of GAS8-AS1 gene (NCBI Reference Sequence: NR_122031.1) is shown as follows:

| (SEQ ID No. 1) | |
|---|---|
| 1 | acctgcagtc ccagctactg ggcagcctga agcagcagga tggtgtgaac ccaggaggtg |
| 61 | gagcttgcag tgagccgagg tcgcgccacc gcactccagc ctgggccaca cagcgagatt |
| 121 | ccgtcagaat cagttacttt tcgggcacag ccccaggcca cttactgtga gcctttttct |
| 181 | ttctcaacac cacattcccc acagggaaaa cacatttctc acctcaaaag aagacaagac |
| 241 | aacgagcaaa caagaaggag cagcaggagg ggttctgagc cgaggatgcc gggcagacat |
| 301 | gagggagaca cgcacccccg aatccaacca gtgcctcggc acaacgacaa atgtcttcac |
| 361 | gtcacagacc tttagaggct cctgggcaga gcctgaacca gggctcctga ctggtctgtt |
| 421 | tggctcacat ggtgttgaga ttttgccatc actcaatatt cagatttctt ataaatatcc |
| 481 | agatttccag cttctcttgg aaaatcagaa aaaaacagca ctgaactcct aggcccacaa |
| 541 | ggcactcccc agtgaacaga tgaaactgtc ctctgctgcg gggcaggagt ctccaggtca |
| 601 | cccccatccc tccccacctg cctggaccct gaagaagcct tctgagtctg tggctcaacg |
| 661 | tgcgatgtgc agtgcaaggg cctgccccgt agcctgcccc gtaggctgcc ccatagcctg |
| 721 | ccccgtaagc tgccccgtag cctgccccgt aggctgcccc gtaggctcca tggccactgc |
| 781 | cccacaaggc ctgtctccac aggaatggga agcggacagg gagacgggca gcagctcaca |
| 841 | tgctgggaca acgcagtgtt caatccattc tccatccagc agctccagac atctttccag |
| 901 | aacacaaacc tgaccccatc acctctctgc ttagccactg gcttaaactg ccaatggttt |
| 961 | gcctgcatgt aaaataaagc cattctttac cattaaaaaa |

Long non-coding RNA (long non-coding RNA, lncRNA) is a kind of non-coding RNA with a transcript of more than 200 nucleotides in length, it is found in recent researches that, lncRNA is a kind of RNA with important biological functions, participating in various important regulating processes, such as genome imprinting, chromosome silencing, chromatin modification, transcriptional activation, transcriptional interference, and intra-nuclear transport, and playing an important role in regulation and control of life activities, such as cell differentiation and development, gene transcription and translation, genetics and epigenetics. In recent years, more and more authoritative studies confirm that lncRNA plays a role of inhibiting or promoting tumors in the formation and development of tumors, and also plays a very important role in the regulation of tumor cell proliferation, apoptosis, cell cycle, invasion and metastasis, etc.

3.1 Experimental Method

3.1.1 According to the present invention, targeted sequencing of 91 pairs of paired tissues of papillary thyroid carcinoma is carried out with the whole genome exon technique described in Embodiment 1.

3.1.2 A real-time quantitative PCR method is used to detect the gene expression of 87 pairs of RNA samples from thyroid carcinoma tissues and para-carcinoma normal tissues of patients with papillary thyroid carcinoma collected by Zhejiang Provincial Tumor Hospital and the Second People's Hospital of Huai'an (referred to as Zhejiang Queue and Huai'an Queue, respectively in the present invention). The real-time quantitative PCR detection kit includes primers designed for real-time quantitative PCR according to the sequence of the GAS8-AS1 gene: GAS8-AS1-F and GAS8-AS1-R, having sequences of SEQ ID No. 2 and SEQ ID No. 3 shown in the table below.

| Prime | Sequence (5′ to 3′) |
|---|---|
| GAS8-AS1-F | CAACGAGCAAACAAGAAGGAG |
| | (SEQ ID No. 2) |
| GAS8-AS1-R | TGAGCCAAACAGACCAGTCA |
| | (SEQ ID No. 3) |

In one embodiment, GAS8-AS1-F or GAS8-AS1-R may also include a FAM fluorescence label and/or restriction endonuclease BamHI sites.

In one embodiment, GAS8-AS1-F may also include a sequence GGATCC that flanks the sequence of SEQ ID No.2, while GAS8-AS1-R may also include a sequence GGATCC that flanks the sequence of SEQ ID No.3.

3.1.3 The papillary thyroid carcinoma cell lines GLAG66, NPA and TPC-1 are cultured in vitro, plasmids carrying the lncRNA GAS8-AS1 gene or siRNAs directed against lncRNA GAS8-AS1 are transfected into the above cells, cells are counted at 24 hours and 48 hours after transfection, to observe the influence of the lncRNA GAS8-AS1 gene on the proliferation of papillary thyroid carcinoma cells.

3.2 Experimental Result

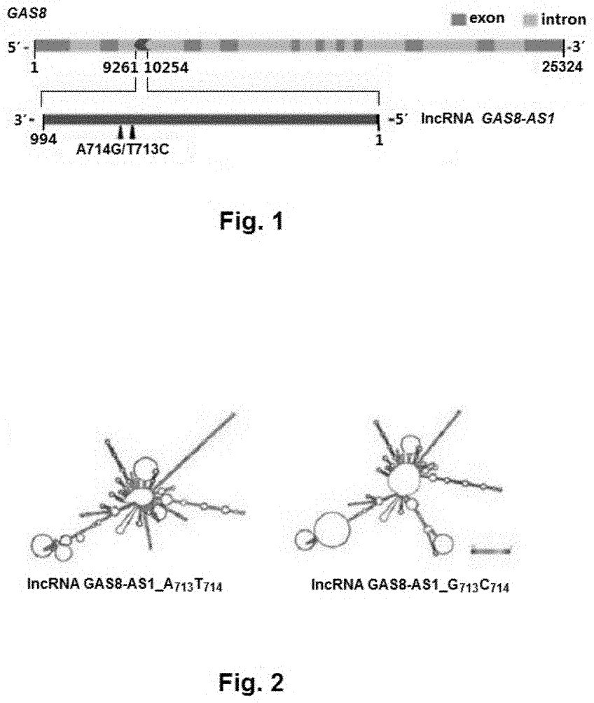

3.2.1 As shown in Table 1, targeted sequencing of 91 pairs of paired tissues of papillary thyroid carcinoma is carried out with the whole genome exon technique, and it is determined that 8 patients carry GAS8-AS1 gene mutation with a mutation rate of 8.8%, and thus determined that GAS8-AS1 gene is a papillary thyroid carcinoma susceptibility gene for the Chinese Han populations. FIG. 1 is a schematic diagram illustrating mutations of lncRNA GAS8-AS1 gene and genes related thereof. The RNA secondary structure of lncRNA GAS8-AS1 predicted by the RNAfold software is as shown in FIG. 2.

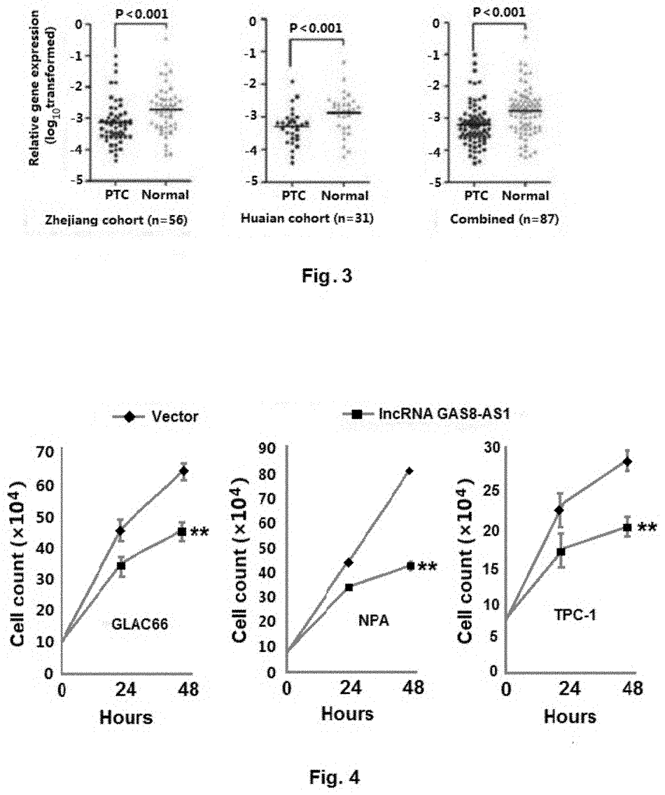

3.2.2 FIG. 3 is a schematic diagram illustrating expression levels of lncRNA GAS8-AS1 in papillary thyroid carcinoma tissues and para-carcinoma normal tissues of papillary thyroid carcinoma patients detected by Zhejiang Queue and Huai'an Queue. As shown in FIG. 3, after detecting the expression of the GAS8-AS1 gene in 87 pairs of papillary thyroid carcinoma tissues and normal tissues from Zhejiang Queue and Huai'an Queue with the real-time quantitative PCR method, it is found that the expression thereof is significantly down-regulated in tumor tissues, notifying the GAS8-AS1 gene is a brand new anti-oncogene of thyroid carcinoma.

3.2.3 FIG. 4 is a schematic diagram illustrating proliferation of papillary thyroid carcinoma cell lines GLAG66, NPA and TPC-1 after transfecting plasmids carrying the lncRNA GAS8-AS1 gene. As shown in FIG. 4, after transfecting plasmids carrying the lncRNA GAS8-AS1 gene, proliferation of GLAG66, NPA and TPC-1 is significantly lower than the carrier of not transfecting plasmids carrying the lncRNA GAS8-AS1 gene. Therefore, lncRNA GAS8-AS1 can significantly inhibit the proliferation of GLAG66, NPA and TPC-1.

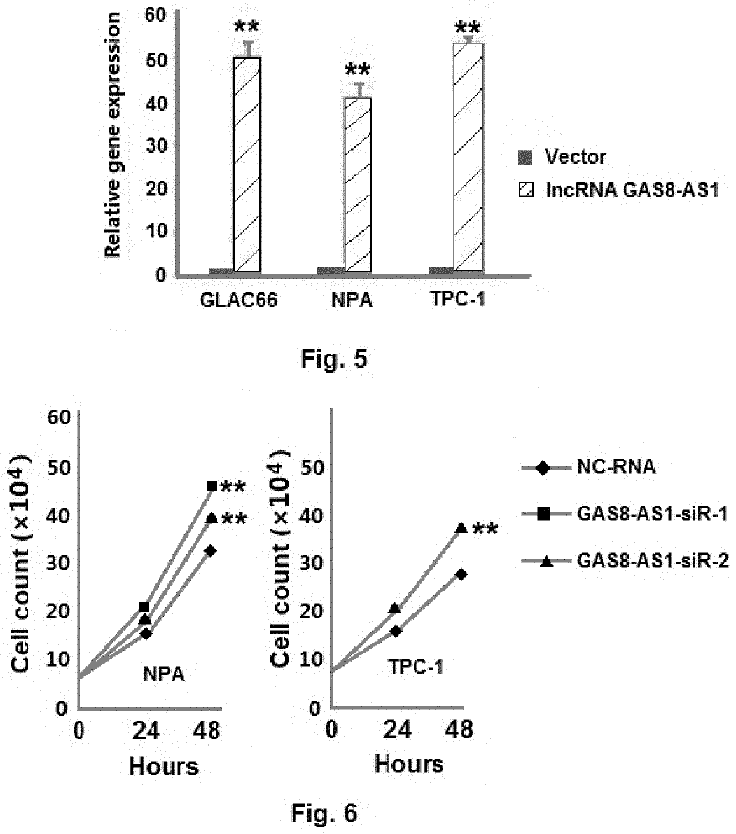

FIG. 5 illustrates expression levels of lncRNA GAS8-AS1 in papillary thyroid carcinoma cell lines GLAG66, NPA and TPC-1 after transfecting plasmids carrying lncRNA GAS8-AS1 gene detected by a real-time and quantitative PCR method. As shown in FIG. 5, the expression levels of lncRNA GAS8-AS1 in GLAG66, NPA and TPC-1 in the carrier of not transfecting plasmids carrying the lncRNA GAS8-AS1 gene are very low, substantially 1.0 or so; the expression levels of lncRNA GAS8-AS1 in GLAG66, NPA and TPC-1 in the carrier of transfecting plasmids carrying the lncRNA GAS8-AS1 gene are about 49, 41, 55, respectively, which is significantly higher than the carrier of not transfecting plasmids carrying the lncRNA GAS8-AS1 gene.

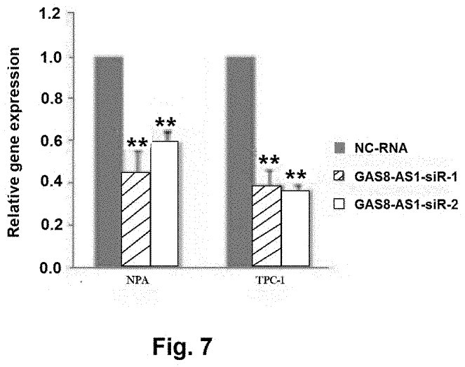

After transfecting GAS8-AS1-siR-1 and GAS8-AS1-siR-2 directed against lncRNA GAS8-AS1, proliferation of papillary thyroid carcinoma cell lines GLAG66 and TPC-1 is increased significantly, as shown in FIG. 6, which indicates that gene silencing of the lncRNA GAS8-AS1 can significantly promote the proliferation of the above cells. After transfecting GAS8-AS1-siR-1 and GAS8-AS1-siR-2 directed against lncRNA GAS8-AS1, expression levels of lncRNA GAS8-AS1 in papillary thyroid carcinoma cell lines GLAG66 and TPC-1 learned by the real-time and quantitative PCR detection are all lower than 0.6, while expression levels if not transfecting GAS8-AS1-siR-1 and GAS8-AS1-siR-2 are 1.0, as shown in FIG. 7.

In summary, overexpression of GAS8-AS1 gene can significantly inhibit the growth of thyroid carcinoma cells in papillary thyroid carcinoma cells cultured in vitro. In contrast, gene silencing of the GAS8-AS1 gene can significantly promote growth of thyroid carcinoma cells.

With the progress of the later research results, the function and action mechanism of GAS8-AS1 in PTC will be clarified gradually that this novel long non-coding RNA not only becomes a diagnosis-related biomarker, but also is expected to become a new PTC therapeutic target so as to improve the clinical PTC treatment effect, having a very important practical significance.

### Embodiment 4: Application of GAS8-AS1 as a Novel Target for Therapies Treating the Papillary Thyroid Carcinoma

4.1 Experiment Method

According to the embodiment 1 disclosed above, the present invention utilizes the whole genome exome trapping techniques to sequence 91 paired tissues (ý thyroid carcinoma tissues and peripheral blood samples) collected from the papillary thyroid carcinoma patients, so as to determine the gene mutations potentially leading to the papillary thyroid carcinoma in patients. GAS81-AS1 is determined as suspected gene of which mutation leads to the papillary thyroid carcinoma in the Chinese Han populations. The paired tissues of papillary thyroid carcinoma patients refer to thyroid carcinoma tissues and peripheral blood samples of the patient, and the Cancer Institute and Hospital of the Chinese Academy of Medical Sciences and Zhejiang Cancer Hospital are entrusted to collect the 91 pairs of tissue samples of papillary thyroid carcinoma patients.

These papillary thyroid carcinoma patients providing 91 pairs of tissues are then grouped into 10 groups, and each group contains at least 10 papillary thyroid carcinoma patients. We used the adeno-associated virus (AAV) as the vector to deliver the long non-coding GAS8-AS1 gene segment into the papillary thyroid carcinoma tissue of the patients. AAV is a well-known tool in gene therapy to deliver the gene segment into tissue of human, which could induce the over expression of the delivered gene segment. Thereafter, GAS8-AS1 is induced to over express before the growth of the papillary thyroid carcinoma cells in the patients are examined.

4.2 Experiment Result

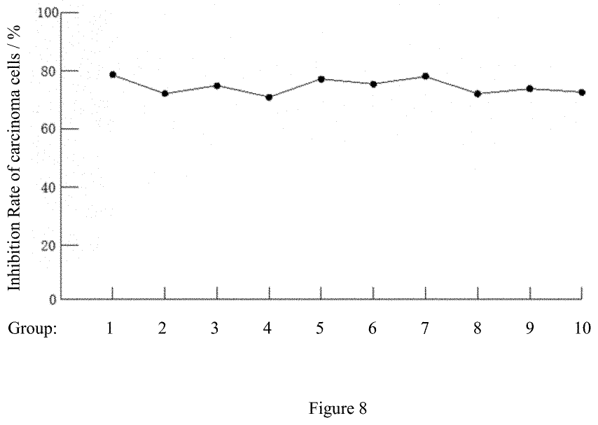

The growth of the papillary thyroid carcinoma cells in the papillary thyroid carcinoma patients whose GAS8-AS1 gene was over expressed are examined. The amount of the papillary thyroid carcinoma cells are quantified, and the inhibition rate (IR) of the carcinoma cells are calculated. Meanwhile, the carcinoma cells of patients who receives only traditional radiotherapy are quantified, and IR of these patients are calculated as well. The comparison of the IRs are compared and reflected in Table 3 and FIG. 8.

TABLE 3
IRs of different therapies for embodiment 4.

| | IR of papillary thyroid | IR of papillary thyroid |
|---|---|---|
| | carcinoma cells of patients | carcinoma cells of patients |
| | who receives GAS8-AS1 | who receives radiotherapy/ |
| | over expression therapy/% | % |
| Group 1 | 78.5 | 44.6 |
| Group 2 | 72.8 | |
| Group 3 | 75.1 | |
| Group 4 | 70.6 | |
| Group 5 | 76.8 | |
| Group 6 | 74.9 | |
| Group 7 | 77.3 | |
| Group 8 | 71.8 | |
| Group 9 | 74.2 | |
| Group 10 | 73.1 | |

As shown in Table 3 and FIG. 8, the IR for the papillary thyroid carcinoma cells of patients who receives GAS8-AS1 over expression therapy are all above 70% and its effectiveness are promising, while the IR for papillary thyroid carcinoma cells of patients who receives traditional radiotherapy is only 44.6%, which is significantly lower than the IR for the papillary thyroid carcinoma cells of patients who receives GAS8-AS1 over expression therapy. As such, the growth of the carcinoma cells of patients who have papillary thyroid carcinoma are significantly inhibited.

Induced over expression of GAS8-AS1 gene in the papillary thyroid carcinoma patients can significantly inhibits the growth of the papillary thyroid carcinoma cells. Therefore, GAS8-AS1 is an effective target for gene therapy of papillary thyroid carcinoma.

As disclosed above, in the papillary thyroid carcinoma cells collected from patients, over expression of GAS8-AS1 gene significantly inhibits the growth of papillary thyroid carcinoma cells in vitro. In contrast, silencing of the GAS8-AS1 gene significantly enhances the growth of the papillary thyroid carcinoma cells in vitro.

With further research, GAS8-AS1's function in the papillary thyroid carcinoma therapy would be further clarified. This novel long non-coding RNA not only serves as a new biomarker for the diagnosis of the papillary thyroid carcinoma, but also provides a target for the gene therapy for the papillary thyroid carcinoma. As such, it significantly improves the treatment for the papillary thyroid carcinoma.

Those described above are just preferred embodiments of the present invention, it should be noted that, various improvements and additions can be made by persons skilled in the art without departing from the method of the present invention, and such improvements and additions shall be deemed to fall within the protection scope of the present invention.

What is claimed is:

1. A method of detecting a long non-coding GAS8-AS1 gene or mRNA in a subject, the method comprising:
obtaining a sample from the subject;
mixing the sample with a forward primer and a reverse primer, the reverse primer including a sequence of CAACGAGCAAACAAGAAGGAG as represented by SEQ ID NO:2 or TGAGCCAAACAGACCAGTCA as represented by SEQ ID NO:3, the sequence is labeled with a FAM fluorescence label; and
amplifying and detecting the long non-coding GAS8-AS1 gene at positions 240-427, located in the intron 2 of human chromosome 16.

2. The method of detecting a long non-coding GAS8-AS1 gene or mRNA in a subject according to claim 1, wherein:
the sample is thyroid carcinoma tissues of the subject.

3. The method of detecting a long non-coding GAS8-AS1 gene or mRNA in a subject according to claim 1, wherein:
the sample is peripheral blood of the subject.

4. A method of diagnosing papillary thyroid carcinoma, the method comprising:
obtaining a sample of papillary thyroid cells from a patient;
hybridizing a nucleic acid molecule having a sequence of
FAM-GGATCCCAACGAGCAAACAAGAAGGAG as represented by
SEQ ID NO: 2
or
GGATCCTGAGCCAAACAGACCAGTCA as represented by SEQ ID
NO: 3
with a long non-coding GAS8-AS1 gene represented by SEQ ID NO:1 in the sample under stringent conditions; and,
diagnosing a patient as having or at an increased risk of developing papillary thyroid carcinoma when expression levels of the gene represented by SEQ ID NO:1 in the sample are at least 40% lower as compared to the expression levels of the gene represented by SEQ ID NO:1 in a healthy person.

5. The method of diagnosing papillary thyroid carcinoma according to claim 4, wherein:
the sample is thyroid carcinoma tissues of the patient.

6. The method of diagnosing papillary thyroid carcinoma according to claim 4, wherein:
the sample is peripheral blood of the patient.

7. A method of treating a subject diagnosed as having papillary thyroid carcinoma, the method comprising:
administering the subject an therapeutic agent comprising a nucleic acid sequence having a long non-coding GAS8-AS1 gene represented by SEQ ID NO:1.


SEQUENCE LISTING (3 sequences)

SEQ ID NO: 1 - DNA, 1000 bp, Homo sapiens
acctgcagtcccagctactgggcagcctgaagcagcaggatggtgtgaacccaggaggtggagcttgcagtgagccgaggtcgcgccaccgcactccagcctgggccacacagcgagattccgtcagaatcagttacttttcgggcacagccccaggccacttactgtgagcctttttctttctcaacaccacattccccacagggaaaacacatttctcacctcaaaagaagacaagacaacgagcaaacaagaaggagcagcaggaggggttctgagccgaggatgccgggcagacatgagggagacacgcacccccgaatccaaccagtgcctcggcacaacgacaaatgtcttcacgtcacagacctttagaggctcctgggcagagcctgaaccagggctcctgactggtctgtttggctcacatggtgttgagattttgccatcactcaatattcagatttcttataaatatccagatttccagcttctcttggaaaatcagaaaaaaacagcactgaactcctaggcccacaaggcactccccagtgaacagatgaaactgtcctctgctgcggggcaggagtctccaggtcacccccatccctccccacctgcctggaccctgaagaagccttctgagtctgtggctcaacgtgcgatgtgcagtgcaagggcctgccccgtagcctgccccgtaggctgccccatagcctgccccgtaagctgccccgtagcctgccccgtaggctgccccgtaggctccatggccactgccccacaaggcctgtctccacaggaatgggaagcggacagggagacgggcagcagctcacatgctgggacaacgcagtgttcaatccattctccatccagcagctccagacatctttccagaacacaaacctgaccccatcacctctctgcttagccactggcttaaactgccaatggtttgcctgcatgtaaaataaagccattctttaccattaaaaaa

SEQ ID NO: 2 - DNA, 21 bp, Artificial Sequence
  Forward primer
caacgagcaaacaagaaggag

SEQ ID NO: 3 - DNA, 20 bp, Artificial Sequence
  Reverse primer
tgagccaaacagaccagtca